(19) United States
(12) Patent Application Publication
(10) Pub. No.: US 2023/0181206 A1
(43) Pub. Date: Jun. 15, 2023

(54) Surgical Instrument

(71) Applicant: G. Hipp & Sohn GmbH, Neuhausen ob Eck (DE)

(72) Inventors: Kai WISSNER, Emmingen-Liptingen (DE); Julian HOFMANN, Neuhausen ob Eck (DE)

(73) Assignee: G. Hipp & Sohn GmbH, Neuhausen ob Eck (DE)

(21) Appl. No.: 17/993,152

(22) Filed: Nov. 23, 2022

(30) Foreign Application Priority Data
Nov. 24, 2021 (DE) 10 2021 130 844.5

(51) Int. Cl. A61B 17/28

(52) U.S. Cl. CPC A61B 17/2833; A61B 2017/2837

(57) ABSTRACT

A surgical instrument has two components hinged to one another, each having an actuating element, and a locking device. The actuating elements can be displaced away from one another by a spreading movement whereby the components are pivoted with respect to one another within a first pivot plane from an initial position into an actuating position. A locking element is in operative connection with the components in a locked position. An unlocking element can be displaced along an unlocking path within a second pivot plane parallel to the first pivot plane in a direction of unlocking. The unlocking element can be brought into operative connection with the locking element, by which the locking element can be transferred into a release position and the components can be pivoted. The unlocking element can be actuated in a direction of unlocking that is the same as the direction of spreading.

### CROSS-REFERENCE TO RELATED APPLICATION

This application claims the priority of German patent application DE 10 2021 130 844.5, filed on Nov. 24, 2021, the contents of which are hereby incorporated by reference in their entirety.

### TECHNICAL FIELD

The invention relates to a surgical instrument for carrying out a surgical procedure, the surgical instrument having a locking device.

### BACKGROUND

Surgical instruments are used in instrumental surgical procedures on or in the body of a patient. Surgical instruments such as forceps, clamps or retractors are used in such operations. The individual components of surgical instruments can be keys, joints, screw connections, shafts, pins, springs, ratchets or bushings.

Manually actuated surgical instruments, the individual components of which are to be fixed in a set position or location after operation, are known. For example, retractors are used to keep the surgical field open through the spreading or distraction, as the case may be, of a wound. However, forceps or clamps, which are actuated for clamping or compressing, as the case may be, a blood channel or for holding a wire and are to be held in a set position, in order to thus provide a clamping force on a lasting basis, are also known. For this purpose, such surgical instruments often have a detent system with a toothing or detents that engage and snap into one another, thus fixing the individual components in the set position. Through appropriate design and prestressing of the individual components of the surgical instrument, the teeth or detents are held in engagement by the prestressing force generated. To release and retract the individual components of the surgical instrument, the detents or the toothing are released from each other by manually applying a release force, and a further spreading movement opens the individual components of the surgical instrument again.

Alternatively, the surgical instruments for unlocking the detent system have an unlocking device with an unlocking element, the actuation of which presses the individual components apart against the direction of the prestressing force, such that the detents are moved apart and the individual components are thereby unlocked. With known wound retractors and clamps, the unlocking device is actuated by a displacement of the unlocking element against the direction of spreading of the surgical instrument.

The hand and finger movements to be performed for releasing the known surgical instruments are unnatural and non-intuitive, since the direction of spreading and the direction of actuation of the unlocking element are in different and opposite directions. In some cases, the one-handed operation of the surgical instrument is not possible, since the unlocking element can only be actuated with a second hand. When the known surgical instruments are used several times within a short period of time, the unnatural hand and finger movements can lead to pain. Moreover, the unnatural grasping, rolling or twisting movements of the fingers or hand create friction between the fingers or hand, as the case may be, and the surgical instrument, which can lead to blistering or pressure sores.

### SUMMARY

The disclosure relates to a surgical instrument for carrying out a surgical procedure. The surgical instrument includes a locking device. The surgical instrument has at least two individual components that are hinged to one another, each having an actuating element. The actuating elements can be displaced away from one another relative to one another in a direction of spreading by a spreading movement. Thereby, the individual components can be pivoted with respect to one another within a first pivot plane and about a first pivot axis from an initial position into an actuating position. The locking device has a locking element that is in operative connection with the individual components in a locked position in such a manner that the individual components can be locked in a displacement position. The locking device has an unlocking element that can be displaced along an unlocking path arranged within a second pivot plane and in a direction of unlocking and can be brought into operative connection with the locking element, by which the locking element can be transferred into a release position and the individual components can be pivoted with respect to one another.

It is considered to be an object of the present disclosure to provide a surgical instrument that can be opened in a simple manner with one hand by an ergonomic hand movement.

This object is achieved in that a setting angle enclosed by the first pivot plane and the second pivot plane has a value in the range from 0° to 90°, and the unlocking element can be actuated along the unlocking path in a direction of unlocking that is the same as the direction of spreading.

It can be provided that the unlocking element can be actuated in a direction of unlocking that is oriented in a manner perpendicular to the first pivot plane. For this purpose, the unlocking element is advantageously arranged on an individual component in such a manner that the unlocking element can be easily performed with a finger that is free during the manual operation of the surgical instrument. When operating a surgical instrument designed as a needle holder or as scissors, for example, the thumb and the ring finger are engaged with the actuating elements designed as scissor eyes. This means, for example, that the middle finger is available for actuating the unlocking element. This enables an intuitive and ergonomic hand movement for transferring the locking element to the release position for unlocking the surgical instrument and for simultaneously spreading the actuating elements of the individual components.

The actuating elements of the individual components may be designed as an eye, a ring or a handle. In the case of a surgical instrument designed as scissors or clamps, the actuating elements are usually arranged at one end of the branches and are operated with one or more fingers. However, it can also be provided that the actuating elements are formed by a section of an individual component. For this purpose, for example, an end region of the individual component can be designed as a graspable handle. Moreover, an embodiment of the actuating elements as a lever fixed to another individual component can be provided.

The locking element can, for example, have a tab-like design and can be rigidly fixed to an individual component. During the spreading movement for actuating the surgical instrument, the actuating elements or the individual components, as the case may be, are moved towards one another, such that the locking element comes into operative connection with the further individual component.

For fixing the individual components relative to one another in a preset position, in an advantageous embodiment, it is provided that the locking element has a first toothing section and at least one individual component has a second toothing section corresponding to the first toothing section. Advantageously, the toothing sections have teeth or detents that engage with on another in a locked position of the locking element. Thus, the individual components can be fixed relative to one another in a set displacement position. In addition to the initial position and the actuation position of the individual components, the displacement position can also be one on an actuation path between the initial position and the actuation position of the individual components. The fact that the teeth engage with one another in the locked position provides a positive-locking connection between the engaged individual component and the locking component, and reduces the risk compared to unintentional release of the displacement position. Thus, safe operation of the surgical instrument is possible.

With respect to the direction of movement of the individual components relative to the locking element during the pivoting movement of the individual components, it can be provided that the toothing sections have longitudinal toothing or transverse toothing. Thereby, in the case of longitudinal toothing, the tooth flanks are arranged in a toothing plane oriented in a manner parallel to the first pivot plane, while in the case of transverse toothing, the tooth flanks are arranged in a toothing plane oriented in a manner perpendicular to the first pivot plane.

In an advantageous implementation, it is provided that, in the locked position, the individual components can be displaced from the initial position to an actuating position, while the displacement of the individual components from an actuating position to an initial position is not possible. In an advantageous embodiment, this is implemented in such a manner that the individual components and the locking element each have a toothing that is free to move in one direction and has a locking effect in the opposite direction. Thus, when force is applied to actuate the surgical instrument in the direction of spreading, it can lock into a next tooth of the serration. Due to the locking effect of the toothing against the direction of spreading, a displacement of the individual components is not possible. Thus, a possibly unintentional release of the surgical instrument can be prevented.

For a particularly secure locking of the individual components in a set displacement position, it is provided in an advantageous embodiment of the surgical instrument that the individual components and the locking element are mounted in a prestressed state, such that a prestressing force is generated by a locking force acting on the individual components by the locking element and the individual components can be locked in the displacement position. By appropriately designing and prestressing the individual components of the surgical instrument, the teeth or detents are held in engagement by the prestressing force generated. The toothing sections can only be separated from one another again by actuating the unlocking element and applying an unlocking force that is opposite to and greater than the prestressing force, such that the individual components are unlocked and movable relative to one another.

For a particularly simple generation of the prestressing force, in an advantageous embodiment, it is provided that the prestressing force is generated by a spring element arranged between at least one individual component and the locking element. Thereby, the spring element is advantageously designed in such a manner that the prestressing force generated enables the toothing of the toothing sections to be held securely. A correspondingly large prestressing force can also enable the low-force actuation of the unlocking element, such that a particularly comfortable and ergonomic manual operation of the surgical instrument is possible.

Depending on the embodiment of the locking device and the unlocking device and depending on the positioning of the spring element between the individual components or between the locking device and an individual component, an advantageous embodiment of the surgical instrument provides that the spring element is designed as a compression spring or as a tension spring. In doing so, different types of springs, such as leaf springs, coil springs or disk springs, can be used.

For a simple redirection of the unlocking force generated by the manual actuation of the unlocking element, it is provided in an advantageous embodiment of the surgical instrument that the unlocking element and the locking element are connected to one another by means of a gear unit. In one simple case, the gear unit is formed by a two-sided lever device, wherein the unlocking element is pivotally fixed to an individual component about a second pivot axis. Thereby, a first gap between the force application point of the manually applied actuating force and the second pivot axis is greater than a second gap between a contact point of the unlocking element and the locking element and the second pivot axis. Due to the lever effect, a greater release force can be transmitted to the clamping element compared to the manually applied actuating force. This allows particularly convenient and easy actuation of the unlocking element.

In an alternative embodiment of the surgical instrument, the unlocking element is engaged with the locking element by means of a spur gear unit. Thereby, one end region of the unlocking element and one end region of the locking element are designed as toothed wheels. The unlocking element and the locking element can each be rotatably displaced about an axis of rotation formed by the toothed wheels. Through the dimensioning of the toothed wheels, the manually applied actuating force can be reduced or increased.

In an advantageous embodiment, it is provided that the individual components and the unlocking element each have at least one sliding surface region. A sliding surface region forms a pair of sliding surfaces with at least one sliding surface region of a further individual component or of the unlocking element. The sliding surface regions bear against one another and slide over one another at least in sliding surface region sections of the sliding surface regions when the surgical instrument is used as intended. The individual components and the unlocking element are made of a metallic or metal-containing material at least in sections that are allocated to the sliding surface regions. Only one of the sliding surface regions of each pair of sliding surfaces has a hard material surface layer, which is connected in a firmly bonded manner to the individual component in the respective sliding surface region. The hard material surface layer has a higher hardness than the metallic or metal-containing material from which the individual components are made. The hard material surface layer is applied by means of suitable processes in the respective friction surface region. By the fact that a hard material surface layer is applied in only one of the two friction surface regions of each pair of friction surfaces, the friction in the friction surface regions of the pair of friction surfaces is reduced. In this manner, the wear of the surgical instrument is reduced and its service life is increased.

In an advantageous embodiment of the surgical instrument, it is provided that only one sliding surface region section of an individual component or the unlocking element of the pair of sliding surfaces has the hard material surface layer. Thereby, the sliding surface region can be designed in such a manner that the sliding surface region section is designed to protrude, such that it can be brought into contact with the sliding surface region of the further individual component. Thus, it is necessary to apply the hard material surface layer only in the sliding surface region section, which makes it possible to reduce the number of shock-sensitive hard material surface layers particularly well and to manufacture a robust surgical instrument.

In an advantageous implementation, it is provided that the individual components having a sliding surface region are made entirely of the metallic or metal-containing material. For example, the movable components of a surgical clamp are made of titanium, wherein the clamp can have handle parts made of plastic that may be fitted over handle parts of clamp elements of the clamp. However, it is also possible that all individual components are made entirely of the metallic or metal-containing material.

The disclosure also relates to a needle holder. The needle holder has two individual components that are hinged to one another, each having an actuating element. The actuating elements can be displaced away from one another relative to one another in a direction of spreading by means of a spreading movement, such that the individual components can be pivoted with respect to one another within a first pivot plane and about a first pivot axis from an initial position into an actuating position. The locking device has a locking element that is in operative connection with the individual components in a locked position in such a manner that the individual components can be locked in a displacement position. The locking device has an unlocking element that can be displaced along an unlocking path arranged within a second pivot plane and in a direction of unlocking and can be brought into operative connection with the locking element, by which the locking element can be transferred into a release position and the individual components can be pivoted with respect to one another. The second pivot plane is aligned in a manner parallel to the first pivot plane and the unlocking element can be actuated along the unlocking path in a direction of unlocking that is aligned in the same direction as the direction of spreading. A spring element arranged between the second individual component and the locking element generates a prestressing force, by means of which the unlocking element is held in a preferred position. This enables the particularly convenient and intuitive operation of the needle holder.

Advantageously, the needle holder in accordance with the disclosure can have the specified features and characteristics of the surgical instrument.

Further advantageous embodiments of the invention are explained with reference to exemplary embodiments shown in the drawings.

### BRIEF DESCRIPTION OF THE DRAWINGS

FIG. 1A and FIG. 1B are schematic illustrations of a surgical instrument designed as a needle holder, each in an exploded perspective view.

FIG. 2A and FIG. 2B are schematic illustrations of an embodiment of a locking device.

FIG. 3A and FIG. 3B are schematic illustrations of a further embodiment of a locking device.

FIG. 4A to FIG. 4F are schematic illustrations of a further embodiment of a locking device.

FIG. 5A and FIG. 5B are schematic illustrations of a further embodiment of a locking device.

FIG. 6 is a schematic illustration of a further embodiment of a locking device.

FIG. 7 is a schematically illustrated view of a surgical instrument and its use.

### DETAILED DESCRIPTION

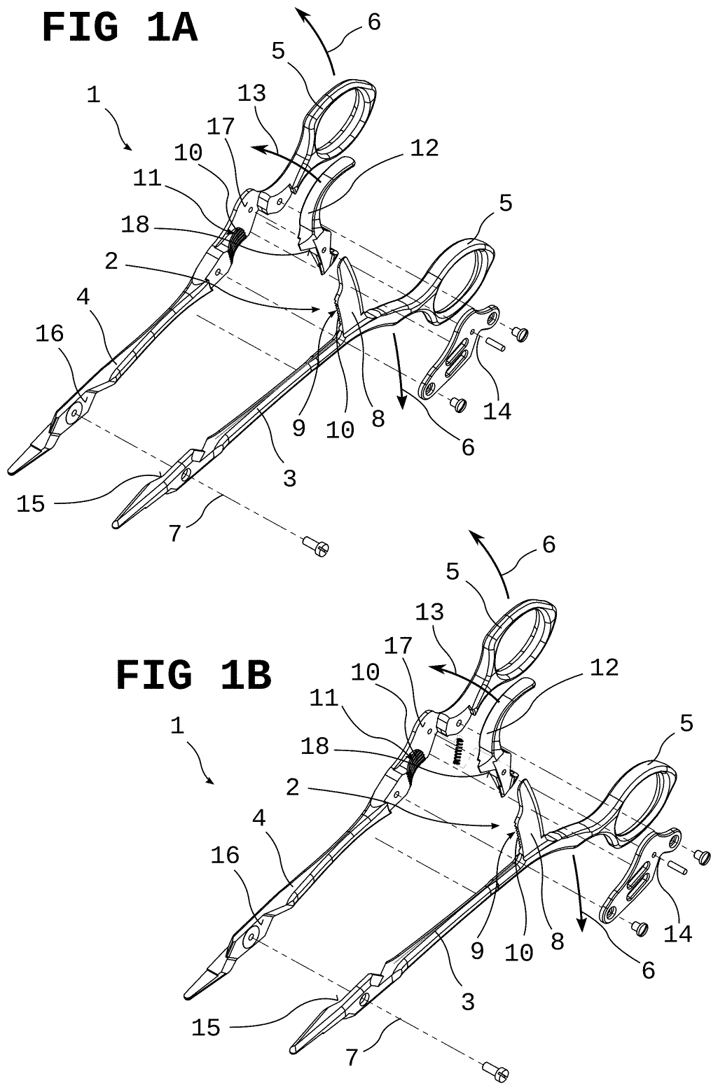

FIG. 1A and FIG. 1B each show a schematic illustration of a surgical instrument 1 designed as a needle holder with a locking device 2 in a perspective view. The surgical instrument 1 has two individual components 3, 4 hinged to one another, each with an actuating element 5. The actuating elements 5 are designed as scissor eyes that can be displaced relative to one another by a spreading movement in a direction of spreading 6. The individual components 3, 4 are pivotally displaceable about a first pivot axis 7 and can be transferred from an initial position to an actuating position.

The locking device 2 has a locking element 8, which is of tab-like design and is rigidly fixed to a first individual component 3. The locking element 8 has a toothing 10 in a first toothing section 9, which is designed as a longitudinal toothing. A second individual component 4 has a second toothing section 11 corresponding to the first toothing section 9 with a longitudinal toothing. In a locked position of the locking element 8, the first toothing section 9 and the second toothing section 11 are engaged such that the individual components 3, 4 are locked in a displacement position. The locking device 2 has an unlocking element 12, which is pivotally displaceable along an unlocking path and in a direction of unlocking 13 about a second pivot axis 14. The unlocking element 12 can be brought into operative connection with the locking element 8, such that the locking element 8 can be transferred into a release position, such that the individual components 3, 4 are unlocked and can be pivoted against one another. The needle holder shown in FIG. 1B has a spring element 19 designed as a compression spring, which is in operative connection with the second individual component 4 and with the unlocking element 12 in such a manner that the unlocking element 12 is held in a preferred position by a prestressing force generated by the spring element 19. Since the unlocking element 12 is in the preferred position both in the initial position and in the actuating position of the individual components 3, 4 and during the spreading movement, the needle holder can be operated particularly conveniently.

The individual components 3, 4 and the unlocking element 12 each have at least one sliding surface region 15, 16, 17, 18. A first sliding surface region 15 of the first individual component 3 forms a first pair of sliding surfaces with a second sliding surface region 16 of the second individual component 4. A third sliding surface region 17 of the second individual component 4 forms a second pair of sliding surfaces with a fourth sliding surface region 18 of the unlocking element 12. Thereby, the first sliding surface region 15 of the first individual component 3 and the fourth sliding surface region 18 of the unlocking element 12 have a hard material surface layer, which is connected in a firmly bonded manner to the respective sliding surface regions 15, 18.

FIG. 2A shows a schematic illustration of the locking device 2, wherein the locking element 8 is shown in a locked position. The locking element 8 is displaceably arranged within the first individual component 3. The first toothing section 9 of the locking element 8 is engaged with the second toothing section 11 of the second individual component 4. Thereby, the first toothing section 9 and the second toothing section 11 each have a toothing 10, which is designed as a transverse toothing with which the tooth flanks of the teeth are oriented in a toothing plane in a manner perpendicular to the pivot plane. The locking element 8 is thereby locked in the locked position, in which a prestressing force is applied by the spring element 19 designed as a compression spring, wherein the prestressing force acts on the locking element 8 and is oriented in the direction of the second toothing section 11. The transfer of the locking element 8 from the locked position to the release position is effected by actuating the unlocking element 12, which is pivotably arranged on the first individual component 3 by means of the second pivot axis 14. This arrangement of the unlocking element 12 forms a two-sided lever device. In FIG. 2B, the locking device 2 is shown in an open state. By actuating the unlocking element 12, the locking element 8 can be transferred to the release position, such that the toothing sections 9,11 are separated from one another.

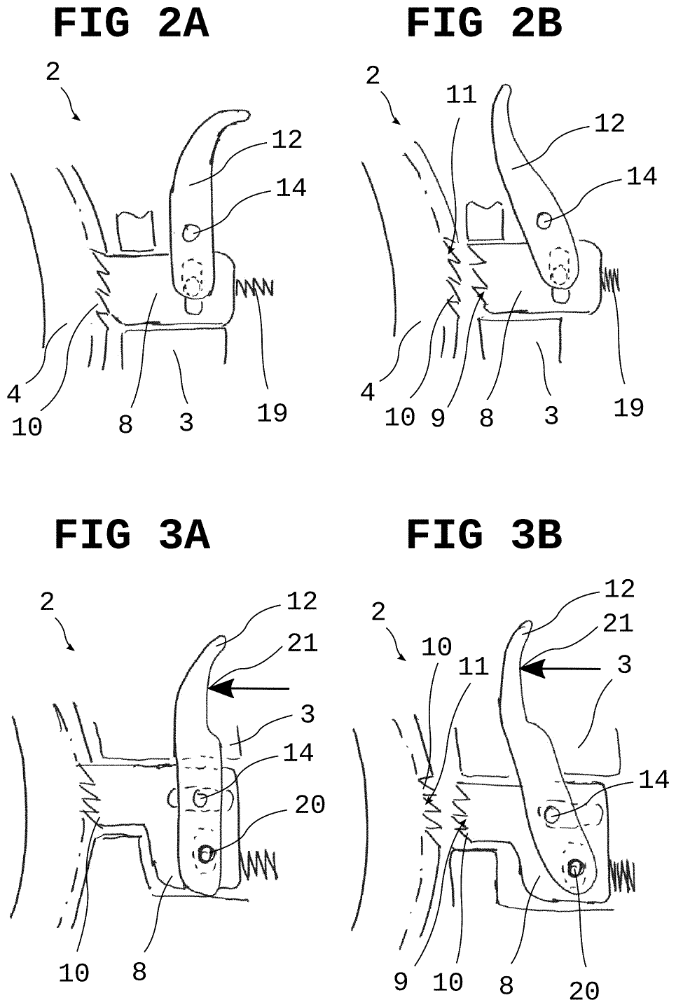

FIGS. 3A and 3B show schematic illustrations of the clamping device 2. In FIG. 3A, the locking device 2 is shown in a locked position of the locking element 8 and in FIG. 3B in a release position of the locking element 8. The embodiments differ from the embodiments shown in FIGS. 2A and 2B in that the unlocking element 12 is arranged to be pivotable about a third pivot axis 20 on the locking element 8 in addition to the second pivot axis 14. As a result, a so-called “one-sided lever device” is formed, by which the locking of the toothings 10 of the first toothing section 9 and the second toothing section 11 takes place on a path between the third pivot axis 20 and a force application point 21 of the manually applied actuating force.

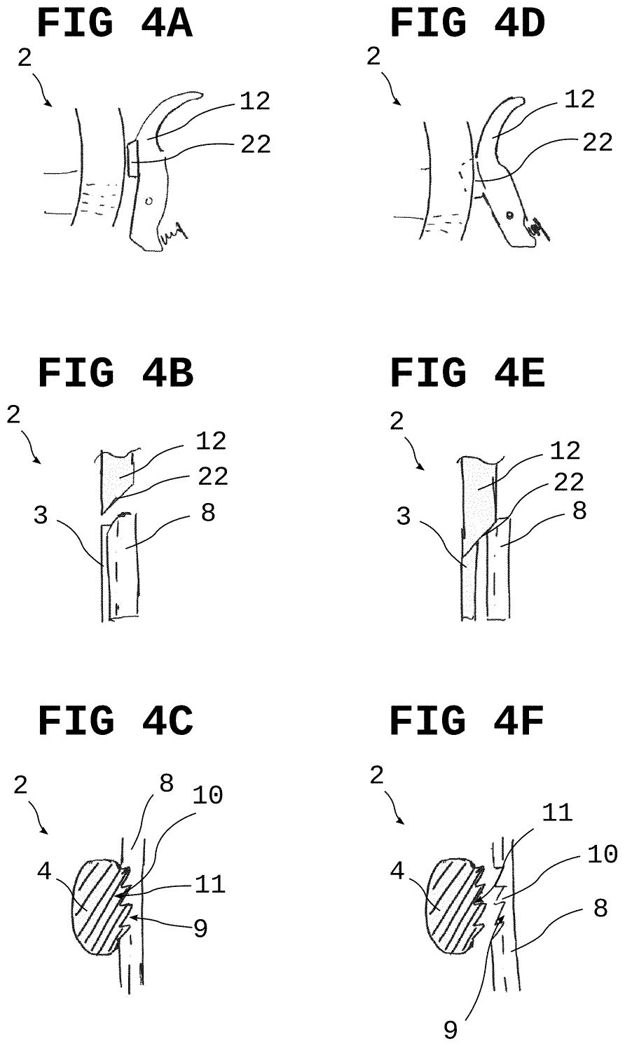

FIGS. 4A to 4F show schematic illustrations of a further embodiment of the clamping device 2. In FIG. 4A, the locking device 2 is shown in a locked position in a side view. The toothing sections 9,11 each have a toothing 10 designed as longitudinal toothing, with which the tooth flanks of the teeth are oriented in a toothing plane in a manner parallel to the pivot plane. In FIG. 4B, the locking device 2 is shown in a top view. Thereby, the unlocking element 12 is not engaged with the first individual component 3 and not with the locking element 8. In FIG. 4C, the locking device 2 is shown in a front view. Thereby, the first toothing section 9 of the locking element 8 is engaged with the second toothing section 11 of the second individual component 4. In FIGS. 4D to 4F, the locking device 2 is shown in an unlocked state, wherein the locking element 8 is transferred to a release position. The unlocking element 12 is in engagement with the locking element 8. Through the wedge-shaped configuration of an engagement section 22 of the unlocking element 12 shown in FIG. 4E and through a further displacement of the unlocking element 12 towards the locking element 8, the locking element 8 slides on the wedge-shaped engagement section 22 of the unlocking element 12, such that the contact between the locking element 8 and the first individual component 3 is released. In FIG. 4F, the locking component 8 is shown in a release position, wherein the first toothing section 9 of the locking component 8 is released from the second toothing section 11 of the second individual component 4.

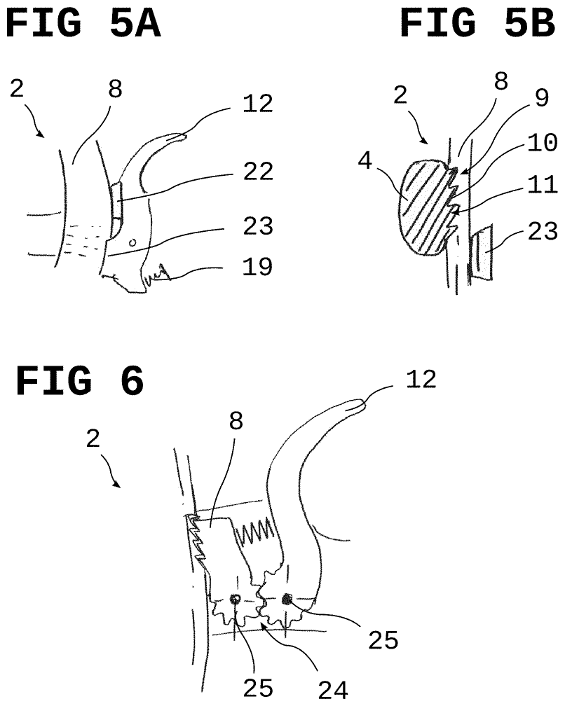

In FIGS. 5A and 5B, a further embodiment of the locking device 2 is shown in a locked position of the locking element 8. Thereby, the unlocking element 12 has, in addition to the engagement section 22, a hold-down section 23 that, in the locked position of the locking element 8, engages around the locking element 8 in sections, such that the first toothing section 9 of the locking element 8 is held in particularly secure engagement with the second toothing section 11 of the second individual component 4. The spring force applied by the spring element 19 holds the unlocking element 12 in a preferred position.

In FIG. 6, a further embodiment of the locking device 2 is shown, wherein the locking element 8 is shown in a locked position. The unlocking element 12 is engaged with the locking element 8 by means of a gear unit 24 designed as a spur gear unit. Thereby, an end region of the unlocking element 12 and an end region of the locking element 8 are formed as toothed wheels, wherein the unlocking element 12 and the locking element 8 are arranged such that they can pivot about respective longitudinal axes 25 of the toothed wheels.

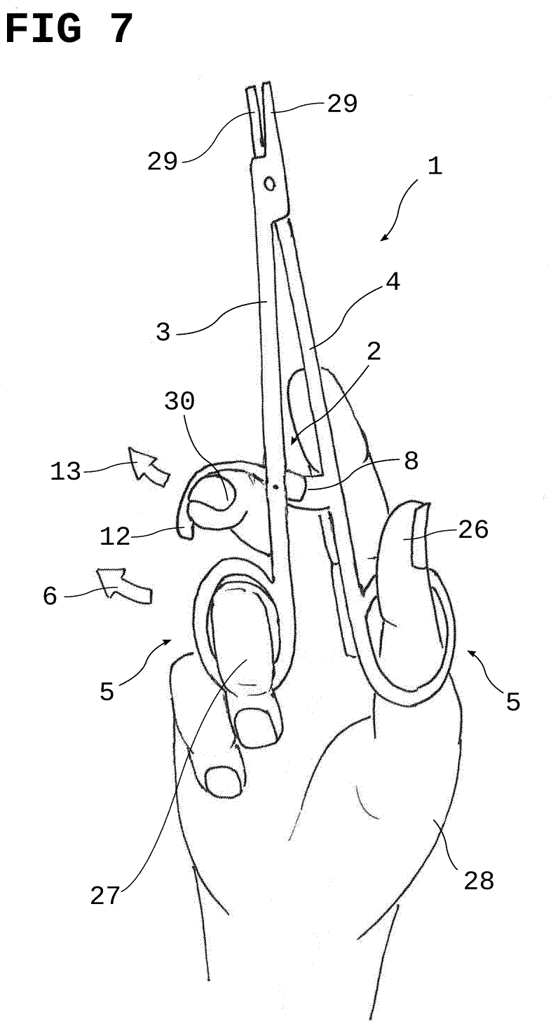

FIG. 7 schematically shows a surgical instrument 1 with two individual components 3, 4, each with an eye-shaped actuating element 5. By means of a spreading movement of a thumb 26 and a ring finger 27 of a hand 28 of an operator of the surgical instrument 1, the actuating elements 5 are moved away from one another, causing manipulation sections 29 of the individual components 3, 4 to move towards one another. In order to be able to carry out the spreading movement, it is necessary to release a locking element 8 of a locking device 2 by actuating an unlocking element 12 of the locking device 2. With the surgical instrument 1 shown in FIG. 7, the unlocking element 12 is actuated by a middle finger 30 of the hand 28. Thereby, the locking device 2 is designed in such a manner that a direction of spreading 6 and a direction of unlocking 13 are in the same direction. In this manner, a particularly ergonomic actuation of the surgical instrument 1 is possible.

In the illustrations in FIGS. 1A to 7, only individual elements of a plurality of similar elements are marked with a reference sign as an example.

1.-14. (canceled)

15. A surgical instrument (1) for carrying out a surgical procedure, comprising:
at least two individual components (3, 4) that are hinged to one another, each having an actuating element (5),
wherein the actuating elements can be displaced away from one another relative to one another in a direction of spreading (6) by a spreading movement, whereby the individual components (3, 4) can be pivoted with respect to one another within a first pivot plane and about a first pivot axis (7) from an initial position into an actuating position; and
a locking device (2),
wherein the locking device (2) has a locking element (8) that is, in a locked position, in operative connection with the individual components (3, 4) in such a manner that the individual components (3, 4) can be locked in a displacement position,
wherein the locking device (2) has an unlocking element (12) that can be displaced along an unlocking path arranged within a second pivot plane and in a direction of unlocking (13) and can be brought into operative connection with the locking element (8), by which the locking element (8) can be transferred into a release position and the individual components (3, 4) can be pivoted relative to one another,
wherein a setting angle enclosed by the first pivot plane and the second pivot plane has a value in a range from 0° to 90°, and the unlocking element (12) can be actuated along the unlocking path in a direction of unlocking (13) that is the same as the direction of spreading (6).

16. The surgical instrument (1) according to claim 15,
wherein the second pivot plane is aligned parallel to the first pivot plane and the unlocking element (12) can be actuated along the unlocking path in a direction of unlocking (13) aligned in the same direction as the direction of spreading (6).

17. The surgical instrument (1) according to claim 15,
wherein the locking element (8) has a first toothing section (9) and at least one individual component (3, 4) has a second toothing section (11) corresponding to the first toothing section (9).

18. The surgical instrument (1) according to claim 15, wherein, in the locked position, the individual components (3, 4) can be displaced from the initial position into an actuating position.

19. The surgical instrument (1) according to claim 15,
wherein the individual components (3, 4) and the locking element (8) are mounted in a prestressed state, such that a prestressing force is generated by a locking force acting on the individual components (3, 4) by the locking element (8) and the individual components (3, 4) can be locked in the displacement position.

20. The surgical instrument (1) according to claim 19,
wherein the prestressing force is generated by a spring element (19) arranged between at least one individual component (3, 4) and the locking element (8).

21. The surgical instrument (1) according to claim 20,
wherein the spring element (19) is designed as a compression spring or as a tension spring.

22. The surgical instrument (1) according to claim 15,
wherein the unlocking element (12) and the locking element (8) are connected to one another via a gear unit (24).

23. The surgical instrument (1) according to claim 15,
wherein the individual components (3, 4) and the unlocking element (12) each have at least one sliding surface region (15, 16, 17, 18),
wherein a sliding surface region (15, 16, 17, 18) forms a pair of sliding surfaces with at least one sliding surface region (15, 16, 17, 18) of a further individual component (3, 4) or of the unlocking element (12),
wherein the sliding surface regions (15, 16, 17, 18) bear against one another at least in sliding surface region sections of the sliding surface regions (15, 16, 17, 18) when the surgical instrument (1) is used as intended,
wherein the individual components (3, 4) and the unlocking element (12) are made of a metallic or metal-containing material at least in sections that are allocated to the sliding surface regions (15, 16, 17, 18),
wherein only one of the sliding surface regions (15, 16, 17, 18) of each pair of sliding surfaces has a hard material surface layer, which is connected in a firmly bonded manner to the individual component (3, 4) in the respective sliding surface region (15, 16, 17, 18).

24. The surgical instrument (1) according to claim 23,
wherein only one sliding surface region section of an individual component (3, 4) or the unlocking element (12) of the pair of sliding surfaces has the hard material surface layer.

25. The surgical instrument (1) according to claim 23,
wherein the individual components (3, 4) having a sliding surface region (15, 16, 17, 18) are made entirely of the metallic or metal-containing material.

26. A needle holder,
wherein the needle holder has two individual components (3, 4) that are hinged to one another, each having an actuating element (5),
wherein the actuating elements (5) can be displaced away from one another relative to one another in a direction of spreading (6) by a spreading movement, such that the individual components (3, 4) can be pivoted with respect to one another within a first pivot plane and about a first pivot axis (7) from an initial position into an actuating position,
wherein a locking device (2) has a locking element (8) that is in operative connection with the individual components (3, 4) in a locked position such that the individual components (3, 4) can be locked in a displacement position,
wherein the locking device (2) has an unlocking element (12) that can be displaced along an unlocking path arranged within a second pivot plane and in a direction of unlocking (13) and can be brought into operative connection with the locking element (8), by which the locking element (8) can be transferred into a release position and the individual components (3, 4) can be pivoted relative one another,
wherein the second pivot plane is aligned parallel to the first pivot plane and the unlocking element (12) can be actuated along the unlocking path in a direction of unlocking (13) that is aligned in the same direction as the direction of spreading (6), and
wherein a spring element (19) arranged between a second of the individual components (4) and the locking element (8) generates a prestressing force, by which the unlocking element (8) is held in a preferred position.
